(19) United States
(12) Patent Application Publication
(10) Pub. No.: US 2015/0164812 A1
(43) Pub. Date: Jun. 18, 2015

(54) Porous Tablets As Carriers For Liquid Formulations

(71) Applicant: Veloxis Pharmaceuticals A/S, Horsholm (DK)

(72) Inventors: Per HOLM, Vanlose (DK); Jannie E. HOLM, Valby (DK); Thomas RUHLAND, Roskilde (DK); Simon DALSGAARD, Copenhagen V (DK)

(21) Appl. No.: 14/635,950

(22) Filed: Mar. 2, 2015

Related U.S. Application Data
(63) Continuation of application No. 11/631,180, filed on Aug. 19, 2008, now abandoned.

(30) Foreign Application Priority Data
Jun. 28, 2004 (DK) PA200401011

(51) Int. Cl. A61K 9/20; A61K 31/496; A61K 31/216; A61K 31/366; A61K 31/436; A61K 31/40

(52) U.S. Cl. CPC A61K 9/2095; A61K 31/436; A61K 31/40; A61K 31/216; A61K 31/366; A61K 31/496; A61K 9/2009

(57) ABSTRACT

A novel tablet product that in an easy, flexible and reproducible manner can be loaded with a relatively high amount of a pharmaceutically acceptable liquid formulation e.g. carrying a therapeutically, prophylactically and/or diagnostically active substance. The novel loadable tablet product may be produced in large-scale batches and stored until use and each batch or sub-batch may be loaded with the same or different pharmaceutically acceptable liquid formulations and/or active substances. A loadable tablet according to the invention has a porosity of 30% v/v or more. The invention also provides tablets that have been loaded with such a liquid formulation as well as a method for the preparation thereof.

### FIELD OF THE INVENTION

The present invention relates to a novel tablet product that in an easy, flexible and reproducible manner can be loaded with a relatively high amount of a pharmaceutically acceptable liquid formulation e.g. carrying a therapeutically, prophylactically and/or diagnostically active substance. The novel tablet product may be produced in large-scale batches and stored until use and each batch or sub-batch may be loaded with the same or different pharmaceutically acceptable liquid formulations and/or active substances. The invention also provides tablets that have been loaded with such a liquid formulation as well as a method for the preparation thereof.

The invention provides a means for obtaining tablets comprising an active substance together with a suitable and relatively high amount of a liquid that influences the accessibility of the active substance e.g. to be released and/or absorbed upon oral administration.

### BACKGROUND OF THE INVENTION

Many drug substances have and it is expected that many of the future drug substances will have undesired properties especially with respect to e.g. water solubility and to oral bioavailability. Therefore, novel technologies, which enable especially therapeutically and/or prophylactically active substances to be delivered to the body in a relatively easy manner and at the same time enables the desired therapeutic and/or prophylactic response, is highly needed.

In the pharmaceutical area it is common to prepare pharmaceutical compositions comprising one or more active substances and various excipients. One reason for preparing such pharmaceutical compositions is to manipulate the availability of the active compound after ingestion of the pharmaceutical composition.

For the preparation of pharmaceutical composition for oral administering the active substances are often incorporated into an agglomerated preparation in order to provide the active compounds in a form that may be pressed into tablets or filled into capsules.

Beside providing the active substance in a form that may be pressed into tablets, agglomerates may also be designed to secure a desired availability of the active compound after ingestion of a pharmaceutical composition containing said granule.

The enhancement of oral bioavailability of poorly water soluble drugs as well as providing a fairly water soluble drug in a sustained release form remain one of the most challenging aspects of drug development and further development of the agglomeration techniques may provide valuable tools for these aspects.

One commonly used technique for granulation is a wet granulation, where a mixture of powders including the active compound is mixed with a liquid, usually an aqueous liquid, under mechanical influence for the preparation of granules. Usually the granules prepared by wet granulation are dried before use.

Melt agglomeration and controlled agglomeration are techniques for agglomeration of an active compound, essentially performed by melting a pharmaceutical acceptable vehicle such as an oil or an oily-like material, dissolution or dispersion of one or more active compounds in the melted vehicle and deposition of the thus prepared mixture on a particulate material, the filler, and subsequently the particles adhere to each other and form agglomerates.

In WO 03/004001 (by the present inventors) is described the novel technique of controlled agglomeration by which it is possible to load a particulate material with a relatively high amount of an oil or an oily-like material. The technique is based on a process that involves spraying of a carrier composition containing the oil or oily-like material onto a particulate material. The process conditions enable the particulate material to be loaded with a relatively high amount of the oil or oily-like material. Normally, the process involves heating of the carrier composition and maintaining the temperature of the carrier composition during application. As the application is performed by spraying, strict temperature control of the spraying equipment is a requirement in order to avoid problems relating to clotting of the spray nozzle etc.

### DESCRIPTION OF THE INVENTION

The present inventors have now found a much more simple solution. They have found that it is possible to prepare a tablet solely containing inert pharmaceutically acceptable excipients (although in some cases it may be suitable also to incorporate an active substance therein) and when the tablet is subjected to a pharmaceutically acceptable liquid formulation e.g. containing the active substance, the tablet will due to its porosity—suck the liquid formulation into the tablet. Most surprising this loading of an inert tablet takes place within a relatively short period of time and is reproducible, i.e. the same amount of liquid formulation is sorbed when the same type and size of tablet and liquid formulation is used (see the examples herein). To the best of the inventors' knowledge, inert tablets with the above-mentioned properties have not been recognized or used before in the pharmaceutical field to load tablets with liquids e.g. containing an active substance.

WO 00/38655 (Alza Corporation) describes a dosage form comprising porous particles. The dosage form may be in the form of tablets that are prepared by mixing porous particles with a liquid carrier such as propylene glycol. However, in contrast to the present invention, this document does not describe inert tablets that have the ability to sorb a liquid active substance or a lipophilic medium containing one or more active substances in a reproducible manner and that results in a high load of liquid.

EP-A-0 001 247 relates to preparations of nifedipine for oral administration in the form of a solution of nifedipine in a polyethylene glycol carried on a pharmaceutically acceptable porous carrier or a noncrystalline dispersion of nifedipine in polyvinylpyrrolidone. No inert loadable tablets are described.

U.S. Pat. No. 6,399,591 (Yung-Shin Pharmaceutical Ind. Co. Ltd.) relates to blank tablets that includes an absorbent, a disintegrant, a lubricant, and a diluent or a binder, or a mixture of a diluent and a binder. An active ingredient in liquid form is introduced into the blank tablet to produce a pharmaceutical composition. However, the examples show that only a load of about 13% w/w is obtainable.

The tablets provided by the present invention can be loaded with any type of active substance as well as they can be designed to any type of release of the active substance.

The loading of the inert tablets is dependent on the type and nature of the pharmaceutically acceptable excipients that are contained in the tablet. The critical parameter is, however, not only the properties of the pharmaceutically acceptable excipients contained in the tablet, but also the properties of the tablet itself. To this end, the most critical properties are the ability of the tablet i) to sorb a pharmaceutically acceptable liquid formulation in a sufficient amount, ii) to maintain the amount sorbed during storage without any sweating of the liquid formulation from the surface of the tablet, and iii) to release the active substance once the tablet is subject to an in vitro dissolution test and/or is administered orally to a subject such as an animal including a human.

To fulfill these requirements, the present inventors have identified that the critical property of the tablets to be loaded is the porosity of the tablet. Accordingly, in one aspect, the present invention relates to a loadable tablet having a porosity of 30% v/v or more as a pharmaceutical carrier composition for a pharmaceutically acceptable liquid formulation. The normal tablets used within the pharmaceutical field have a porosity that is much lower. One of the reasons to avoid very porous tablets are that such tablets do not have sufficient robustness to enable the normal handling of tablets during packaging and storage, i.e. they are excepted not to fulfill the pharmacopoeia requirements with respect to hardness and friability.

Porosity is defined as the volume ratio between the voids in the tablet and the total volume of the tablet according to Equation 1 in the Examples herein.

### Loadable Tablets

In the present context the term “inert tablet” is used to denote a tablet that solely contains ingredients that normally are regarded as inert with respect to therapeutic effect. More specifically, such a tablet contains pharmaceutically acceptable excipients selected from the group consisting of fillers, diluents, binders, lubricants, glidants etc. Additives such as, e.g., pH adjusting agents, buffering agents, enhancers, wetting agents, solubilizing agents, surfactants, antioxidants etc. The term “loadable tablet” used in the present context denotes an “inert tablet” as defined above, but further having a porosity of at least about 30% v/v in order to enable a suitable loading with a liquid. However, in some cases it may be of interest to include an active substance in such a tablet and, accordingly, the term “loadable tablet” also includes such cases. In a preferred embodiment, the tablets are “inert and loadable”, i.e. without any content of active substance before loading.

However, as shown in the Examples herein, the present inventors have found that it is possible to load tablets having a high porosity with a pharmaceutically acceptable liquid, preferably containing one or more therapeutically, prophylactically and/or diagnostically active substances (in the following abbreviated “active substance”). The loaded tablets are sufficiently robust to withstand the normal handling of tablets during further processing (e.g. coating), packaging, storage etc., i.e. they fulfill the pharmacopoeial requirements with respect to hardness and friability.

In a specific embodiment a loadable tablet according to the invention results—when tested as described herein—in a loading of the tablet with at least 20% w/w such as, e.g., at least 25% w/w or at least 30% w/w of corn oil (based on the total weight of the solid dosage form upon loading). Such a test ensures that the tablet has the ability of sorbing a liquid formulation that is suitable for use in the preparation of tablets.

As mentioned above, the loadable tablets according to the invention are sufficiently robust to withstand the normal handling of tablets, i.e. they have a hardness of 20 N or more such as, e.g., about 25 N or more, about 30 N or more, about 35 N or more, about 40 N or more, about 45 N or more or about 50 N or more.

Furthermore, the tablets according to the invention have a friability of about 5% or less such as, e.g., about 4% or less, about 3% or less, about 2% or less such as about 1% or less.

As mentioned above, the loadable tablets according to the invention comprise one or more pharmaceutically acceptable excipients. It is however, important that a least one pharmaceutically acceptable excipient has the right properties with respect to providing a tablet with a porosity of 30% v/v or more and that this excipient is present in a sufficient amount so that the tablet obtained also has the desired porosity. Such pharmaceutically acceptable excipients are in some cases herein denoted “pharmaceutically acceptable, porosity providing excipients”. To this end, the present inventors have found that if the pharmaceutically acceptable excipient is manufactured into tablets together with at the most 50% w/w of lactose or other pharmaceutically acceptable excipients used for direct compression such as, e.g., Emcompress, and the tablets obtained have a porosity of 30 vol % or more, then the pharmaceutically acceptable excipient is suitable for use in the present context. The quality of lactose is for direct compression.

In the loadable tablets the sum of pharmaceutically acceptable excipients that have the above-mentioned property (i.e. fulfils the above-mentioned test) corresponds to at least 50% w/w such as, e.g. at least 55% w/w, at least 60% w/w, at least 65% w/w, at least 70% w/w, at least 80% w/w, at least 90% w/w, at least 95% w/w or at least 98% w/w such as e.g. 100% w/w of the total weight of the tablet.

In preferred aspects, the one or more porosity providing excipients are present in a concentration of about 50% w/w or more such as, e.g., about 60% w/w or more such as, e.g., about 70% w/w or more, about 80% w/w or more, about 90% w/w or more or about 95% w/w or more in the tablet.

Moreover, it is contemplated that the specific surface area (BET surface area) of the porosity providing excipient should be relatively large such as, e.g., at least 50 m²/g as measured by gas adsorption.

In the following is given a list of pharmaceutically acceptable excipients that have suitable properties that enable providing a loadable tablet according to the invention. The individual pharmaceutically acceptable excipients may be used alone or in combination provided that the overall aim is obtained with respect to porosity.

To this end, it should be noted that the tablets are compressed into tablets by use of a certain compression force. However, the compression force may not be so low that the requirements with respect to hardness and friability of the tablets are compromised, i.e. these requirements ensure that the tablets are sufficiently robust.

Suitable pharmaceutically acceptable excipients that can be used to obtain tablets having a porosity of 30% v/v or more are selected from the group consisting of metal oxides, metal silicates, metal carbonates, metal phosphates, metal sulfates, sugar alcohols, sugars and cellulose and cellulose derivatives. The metal is typically selected from the group consisting of sodium, potassium, magnesium, calcium, zink, aluminium, titanium and silicium.

A suitable metal oxide for use according to the invention may be selected from the group consisting of magnesium oxide, calcium oxide, zink oxide, aluminium oxide, titanium dioxide including Tronox A-HP-328 and Tronox A-HP-100, silicium dioxides including Aerosil, Cab-O-Sil, Syloid, Aeroperl, Sunsil (silicon beads), Zeofree, Sipernat, and mixtures thereof.

In a specific embodiment, the metal oxide is a titanium dioxide or a silicium dioxide or mixtures thereof.

The silicates can be divided in the following groups:
Swelling clays of the smectite type e.g. bentonite, veegum, laponite.
Hydrous aluminium silicates or alkaline earths. Neusilin belongs to this group and is based on synthetic polymerisation (magnesium aluminium metasilicate).
Silicon dioxides are subdivided into porous and nonporous silicas
   Nonporous colloidal silicas e.g. Aerosil (fumed silicas)
   Porous silicas gels e.g. Syloid, Porasil, Lichrosorp
   Others e.g. Zeopharm S170, Zeopharm 6000, Aeroperl 300

Accordingly, a loadable tablet according to the invention may contain a metal oxide that is a non-porous silicate including fumed silicas of the Aerosil type, and/or a porous silicate including e.g. Syloid, Porasil and Lichrosorp.

In other embodiments the pharmaceutically acceptable excipient for use according to the invention is a metal silicate selected from the group consisting of sodium silicate, potassium silicate, magnesium silicate, calcium silicate including synthetic calcium silicate such as, e.g., Hubersorp, zink silicate, aluminum silicate, sodium aluminosilicate such as, e.g., Zeolex, magnesium aluminum silicate, magnesium aluminum metasilicate, aluminium metasilicate, Neusilin SG2 and Neusilin US2 and mixtures thereof.

The metal silicate may also be a swelling clay of the smectite type selected from the group consisting of bentonite, veegum and laponite, and/or the metal silicate is selected from alkaline earth metal silicates and aluminum silicates included magnesium aluminum metasilicate. In a specific embodiment the metal silicate is Neusilin.

As mentioned above a suitable pharmaceutically acceptable excipient may be a metal carbonate such as a carbonate selected from the group consisting of sodium carbonate, sodium hydrogen carbonate, potassium carbonate, potassium hydrogen carbonate, calcium carbonate, magnesium carbonate, zink carbonate and aluminum carbonate, and mixtures thereof.

Other metal salt suitable for use according to the invention are metal phosphates selected from the group consisting of sodium phosphate, disodium hydrogen phosphate, sodium dihydrogen phosphate, potassium phosphate, dipotassium hydrogen phosphate, potassium dihydrogen phosphate, calcium phosphate, magnesium phosphate, zink phosphate and aluminum phosphate.

More specifically, the pharmaceutically acceptable excipient may be a calcium phosphate selected from the group consisting of dibasic anhydrous calcium phosphate, dibasic dihydrate calcium phosphate, and tribasic calcium phosphate.

The dibasic anhydrous calcium phosphate is typically selected from the group consisting of A-Tab, calcium monohydrogen phosphate, calcium orthophosphate, Di-Cafos AN, dicalcium orthophosphate, E341, Anhydrous Emcompress, Fujicalin, phosphoric acid calcium salt (1:1), and secondary calcium phosphate, and mixtures thereof. The dibasic dihydrate calcium phosphate may be selected from the group consisting of Cafos, calcium hydrogen orthophosphate dihydrate, calcium monohydrogen phosphate dihydrate, Calipharm, Calstar, Di-Cafos, dicalcium orthophosphate, DI-TAB, Emcompress, phosphoric acid calcium salt (1:1) dihydrate, secondary calcium phosphate, Fujiclin SG.

Examples of tribasic calcium phosphates are e.g. hydroxyapatite, phosphoric acid calcium salt (2:3), precipitated calcium phosphate, tertiary calcium phosphate, Tri-Cafos, tricalcium diorthophosphate, tricalcium orthophosphate, tricalcium phosphate, TRI-CAL, WG, TRI-TAB.

Other suitable metal salts are metal sulfates such as, e.g, sodium sulfate, sodium hydrogen sulfate, potassium sulfate, potassium hydrogen sulfate, calcium sulfate, magnesium sulfate, zink sulfate and/or aluminum sulfate.

Examples of suitable calcium sulfates are e.g. calcium sulfate anhydrous including anhydrite, anhydrous gypsum, anhydrous sulfate of lime, Destab, Drierte, E516, karstenite, muriacite, and Snow White or calcium sulfate dihydrate including alabaster, Cal-Tab, Compactrol, Destab, E516, gypsum, light spar, mineral white, native calcium sulfate, precipitated calcium sulfate, satinite, satin spar, selenite, terra alba and USG Terra Alba.

In other embodiments, the pharmaceutically acceptable excipient may be a sugar alcohol selected from the group consisting of sorbitol (such as, e.g., Sorbogem, SPI Pharma), xylitol, mannitol (such as, e.g., Mannogem, SPI Pharma), maltitol, inositol, mannitol (e.g. Pealitol SP 100) and/or it may be a sugar selected from the group consisting of mono-, di- or polysaccharides including saccharose, glucose, fructose, sorbose, xylose, lactose, dextran, dextran derivatives, cyclodextrins.

Cellulose and cellulose derivatives are also suitable pharmaceutically acceptable excipients for the purpose of obtaining tablets having a porosity of 30% v/v or more. Examples include cellulose, microcrystalline cellulose, Celphere, cellulose derivatives including porous cellulose beads: cellulose acetate Celluflow TA-25 and cellulose Celluflow C-25, hydroxypropyl methylcellulose (HPMC), hydroxypropyl cellulose (HPC), methylcellulose, ethylcellulose, sodium carboxymethylcellulose, hydroxyethyl cellulose etc.

### Other Pharmaceutically Acceptable Excipients for Use in a Loadable Tablet According to the Invention

The loadable tablet may of course also contain other pharmaceutically acceptable excipients such as those normally employed in the manufacturing of tablets.

In the present context the terms “pharmaceutically acceptable excipient” are intended to denote any material, which is inert in the sense that it substantially does not have any therapeutic and/or prophylactic effect per se. Such an excipient may be added with the purpose of making it possible to obtain a pharmaceutical, cosmetic and/or foodstuff composition, which have acceptable technical properties.

Examples of suitable excipients for use in a loadable tablet according to the invention include fillers, diluents, disintegrants, binders, lubricants etc. or mixture thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc. Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the tablet. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a loadable tablet of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilising agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance.

Examples on surfactants are given in the following.

Suitable excipients for use in a tablet according to the invention are surfactants such as, e.g., amphiphillic surfactants as those disclosed in WO 00/50007 in the name of Lipocine, Inc. Examples on suitable surfactants are
i) polyethoxylated fatty acids such as, e.g. fatty acid mono- or diesters of polyethylene glycol or mixtures thereof such as, e.g. mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000,
ii) polyethylene glycol glycerol fatty acid esters, i.e. esters like the above-mentioned but in the form of glyceryl esters of the individual fatty acids;
iii) glycerol, propylene glycol, ethylene glycol, PEG or sorbitol esters with e.g. vegetable oils like e.g. hydrogenated castor oil, almond oil, palm kernel oil, castor oil, apricot kernel oil, olive oil, peanut oil, hydrogenated palm kernel oil and the like,
iv) polyglycerized fatty acids like e.g. polyglycerol stearate, polyglycerol oleate, polyglycerol ricinoleate, polyglycerol linoleate,
v) propylene glycol fatty acid esters such as, e.g. propylene glycol monolaurate, propylene glycol ricinoleate and the like,
vi) mono- and diglycerides like e.g. glyceryl monooleate, glyceryl dioleae, glyceryl mono- and/or dioleate, glyceryl caprylate, glyceryl caprate etc.;
vii) sterol and sterol derivatives;

viii) polyethylene glycol sorbitan fatty acid esters (PEG-sorbitan fatty acid esters) such as esters of PEG with the various molecular weights indicated above, and the various Tween® series;
ix) polyethylene glycol alkyl ethers such as, e.g. PEG oleyl ether and PEG lauryl ether;

x) sugar esters like e.g. sucrose monopalmitate and sucrose monolaurate;
xi) polyethylene glycol alkyl phenols like e.g. the Triton® X or N series;
xii) polyoxyethylene-polyoxypropylene block copolymers such as, e.g., the Pluronic® series, the Synperonic® series, Emkalyx®, Lutrol®, Supronic® etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407;
xiii) sorbitan fatty acid esters like the Span® series or Ariacel® series such as, e.g. sorbinan monolaurate, sorbitan monopalmitate, sorbitan monooleate, sorbitan monostearate etc.;
xiv) lower alcohol fatty acid esters like e.g. oleate, isopropyl myristate, isopropyl palmitate etc.;
xv) ionic surfactants including cationic, anionic and zwitterionic surfactants such as, e.g. fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0.1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

Tablets Loaded with a Pharmaceutically Acceptable Liquid

The tablets describe above are designed so that they can be loaded with pharmaceutically acceptable liquid formulation in a concentration of about 20% w/w or more such as, e.g., about 25% w/w or more, about 30% w/w or more (based on the total weight of the solid dosage form upon loading). Accordingly, in another aspect the invention relates to such tablets.

In preferred aspects, the pharmaceutically acceptable liquid formulation is present in a concentration of about 40% w/w or more such as, e.g., about 50% w/w or more or about 60% w/w or more (based on the total weight of the solid dosage form upon loading).

A critical parameter in connection with the loading of the liquid formulation is the viscosity of the liquid formulation. The loading can be performed in any possible manner such as, e.g., by placing the tablets in a suitable container containing the liquid or by spraying the liquid on the tablets in a suitable apparatus such as, e.g., using conventional coating equipment such as coating pan, perforated vessel or fluidized bed. Especially the viscosity of the liquid is important when the liquid formulation is sprayed on the tablets. Accordingly, in a specific embodiment the pharmaceutically acceptable liquid formulation has a viscosity of at the most about 600 mPa sec at a temperature of at the most about 150° C.

Furthermore, the pharmaceutically acceptable liquid formulation normally has a melting point of at least about 0° C. and at the most about 250° C. such as, e.g., about 5° C. or more such as, e.g., about 10° C. or more, about 15° C. or more, about 20° C. or more or about 25° C. or more. The melting point is not very critical as the liquid formulation may be heated or cooled in connection with loading of the tablets with the liquid formulation. The pharmaceutically acceptable liquid formulation can be based on water or it can be based on an organic solvent or an oil or an oily-like material. Surprisingly, the inventors have found that a loadable tablet according to the invention can be dipped into water and upon saturation with water (which takes only a few minutes or less) the tablet appear with a cold, but dry surface, i.e. water and aqueous based liquid can also be employed a suitable pharmaceutically acceptable liquid formulation.

However, the more general applicability is envisaged with respect to loading the tablets with active substances contained in an aqueous or organic based liquid. Such liquids include oil or oily-like materials or pharmaceutically acceptable solvents.

Such oils or oily-like materials may be selected from the group consisting of water, vegetable oils, hydrogenated vegetable oils, and animal oils.

Suitable examples include apricot oil, almond oil, avocado oil, castor oil, coconut fat, cocoa butter, corn oil, cotton seed oil, grape seed oil, jojoba oil, linseed oil, maize oil, olive oil, palm oil, peanut oil, persil oil, poppy seed oil, rape seed oil, sesame oil, soybeen oil, sunflower oil, thistle seed oil, walnut oil, wheat germ oil, beef tallow, lard, tall oil, whale oil, and mixtures thereof.

Other examples are hydrophilic oils or oily-like materials selected from the group consisting of: polyether glycols such as, e.g., polyethylene glycols, polypropylene glycols; polyoxyethylenes; polyoxypropylenes; poloxamers and mixtures thereof, or it may be selected from the group consisting of: xylitol, sorbitol, potassium sodium tartrate, sucrose tribehenate, glucose, rhamnose, lactitol, behenic acid, hydroquinon monomethyl ether, sodium acetate, ethyl fumarate, myristic acid, citric acid, Gelucire 50/13, other Gelucire types such as, e.g., Gelucire 44/14 etc., Gelucire 50/10, Gelucire 62/05, Sucro-ester 7, Sucro-ester 11, Sucro-ester 15, maltose, mannitol and mixtures thereof.

The oil or oily-like material may also be a hydrophobic oil or oily-like material selected from the group consisting of: straight chain saturated hydrocarbons, sorbitan esters, paraffins; fats and oils such as e.g., cacao butter, beef tallow, lard, polyether glycol esters; higher fatty acid such as, e.g., stearic acid, myristic acid, palmitic acid, higher alcohols such as, e.g., cetanol, stearyl alcohol, low melting point waxes such as, e.g., glyceryl monostearate, glyceryl monooleate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, or a mixture thereof.

Suitable polyethylene glycols generally have an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In a specific embodiment, the oil or oily-like material may be a polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

Poloxamers can also be used according to the invention. Examples include Poloxamer 188, Poloxamer 237, Poloxamer 338 or Poloxamer 407 or other block copolymers of ethylene oxide and propylene oxide such as the Pluronic® and/or Tetronic® series. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

In another embodiment, the oil or oily-like material may be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

Moreover or alternatively, the oil or oily-like material may be a mixture of different oils or oily-like materials such as, e.g., a mixture of hydrophilic and/or hydrophobic materials, or a solvent or a semi-solid excipient like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

The pharmaceutically acceptable liquid formulation may also be a dispersion including an emulsion, a microemulsion e.g. a self-microemulsifying drug delivery system (SMEDDS) or a suspension.

Typically the concentration of the pharmaceutically acceptable liquid formulation in the tablet is about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 60% w/w or more or about 70% or more.

The tablets obtained after loading of a loadable tablet with a pharmaceutically acceptable liquid formulation typically fulfill the pharmacopoeia requirements. Thus, a tablet according to the invention typically has a hardness of at least about 20 N and/or a friability of at the most about 5% such as, e.g., at the most about 4%, at the most about 3%, at the most about 2%, at the most about 1% or at the most about 0.5%.

Furthermore, it is contemplated that the loading of the liquid into a loadable tablet of the invention results in a substantially homogeneous distribution of the liquid within the tablet.

Furthermore, the tablets can be designed to release the active substance substantially immediately or in a modified manner. A tablet designed to immediate release typically has a disintegration time of at the most 15 min as tested according to Ph. Eur, whereas a film coated tablet may have a disintegration time of at the most about 30 min. For modified release tablets, the release of the active substance is of importance.

For a plain tablet according to the invention at least 75% of the therapeutically, prophylactically and/or diagnostically active substance is released within 30 min when tested in a dissolution method according to USP.

As mentioned above, a preferred embodiment is a tablet loaded with one or more therapeutically, prophylactically and/or diagnostically active substances.

### Effervescent Tablet Disintegration Formulation Principle

The present inventors have found that disintegration of tablets loaded with lipophilic formulation is not improved by adding a hydrophilic superdisintegrant due to reduced swelling properties of the disintegrant in the lipid environment. In this case a different disintegration principle might be applied based on an effervescent effect. The disintegration of the tablet is improved by the internal release of carbon dioxide. An effervescent tablet formulation is based on a combination of metal carbonates with and acid source. Metal carbonates are such as sodium bicarbonate, sodium carbonate, potassium bicarbonate, potassium carbonate, calcium carbonate, and sodium sesquicarbonate. The acid sources are such as citric acid, sodium dihydrogen citrate, disodium hydrogen citrate, tartaric acid, malic acid, fumaric acid, sodium dihydrogen phosphate, and sodium acid sulfite. The acid component might be excluded in the tablet formulation as the effervescent effect is obtained in-vivo when the tablet is dissolved in the acid gastric juice and reacts with the metal carbonate.

### Coating

The tablet may also be coated with a film coating e.g. for immediate or modified release, an enteric coating, a modified release coating, a protective coating, an anti-adhesive coating etc.

Suitable coating materials are e.g. methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, zein.

Plasticizers and other ingredients may be added in the coating material. The same or different active substance may also be added in the coating material.

### Melt Coating

The hydrophobic surface of a lipid loaded tablet according to the invention might prevent adhesion of a coating polymer applied in aqueous or organic solvent. As alternative, melt coating is suitable using different lipophilic meltable lipids sprayed in melted form and solidified onto tablet surface using conventional coating equipment. Useful melt coating substances are such as, polyglycolised glycerides (Gelucire 50/02, Gelucire 62/05, Gelucire 53/10), polyglyceryl palmitostearate, Glyceryl behenate (Compritol 888 ATO), glyceryl stearate (Precirol WL), glyceryl palmito stearate (Precirol ATO 5), polyglycolised unsaturated glycerides (Labrafil M1944).

### Active Substances

In the present context a therapeutically and/or prophylactically active substance includes any biologically and/or physiologically active substance that has a function on an animal such as, e.g. a mammal like a human. The term includes drug substances, hormones, genes or gene sequences, antigen-comprising material, proteins, peptides, nutrients like e.g. vitamins, minerals, lipids and carbohydrates and mixtures thereof. Thus, the term includes substances that have utility in the treatment and/or preventing of diseases or disorders affecting animals or humans, or in the regulation of any animal or human physiological condition. The term also includes any biologically active substance which, when administered in an effective amount, has an effect on living cells or organisms.

Examples on active substances suitable for use in a tablet according to the invention are in principle any active substance such as, e.g. freely water soluble as well as more slightly or insoluble active substances. Thus, examples on active substances suitable for use are e.g. antibacterial substances, antihistamines and decongestants, anti-inflammatory agents, antiparasitics, antivirals, local anesthetics, antifungals, amoebicidals or trichomonocidal agents, analgesics, antianxiety agents, anticlotting agents, antiarthritics, antiasthmatics, antiarthritic, anticoagulants, anticonvulsants, antidepressants, antidiabetics, antiglaucoma agents, antimalarials, antimicrobials, antineoplastics, antiobesity agents, antipsychotics, antihypertensives, antitussives, auto-immune disorder agents, anti-impotence agents, anti-Parkinsonism agents, anti-Alzheimers' agents, antipyretics, anticholinergics, anti-ulcer agents, anorexic, beta-blockers, beta-2 agonists, beta agonists, blood glucose-lowering agents, bronchodilators, agents with effect on the central nervous system, cardiovascular agents, cognitive enhancers, contraceptives, cholesterol-reducing agents, cytostatics, diuretics, germicidals, H-2 blockers, hormonal agents, hypnotic agents, inotropics, muscle relaxants, muscle contractants, physic energizers, sedatives, sympathomimetics, vasodilators, vasoconstrictors, tranquilizers, electrolyte supplements, vitamins, counterirritants, stimulants, anti-hormones, drug antagonists, lipid-regulating agents, uricosurics, cardiac glycosides, expectorants, purgatives, contrast materials, radiopharmaceuticals, imaging agents, peptides, enzymes, growth factors, etc.

Specific examples include e.g.

Anti-inflammatory drugs like e.g. ibuprofen, indometacin, naproxen, nalophine;

Anti-Parkinsonism agents like e.g. bromocriptine, biperidin, benzhexol, benztropine etc.

Antidepressants like e.g. imipramine, nortriptyline, pritiptyline, etc.

Antibiotics like e.g. clindamycin, erythomycin, fusidic acid, gentamicin, mupirocine, amfomycin, neomycin, metronidazol, sulphamethizole, bacitracin, framycetin, polymyxin B, acitromycin etc,

Antifungal agents like e.g. miconazol, ketoconaxole, clotrimazole, amphotericin B, nystatin, mepyramin, econazol, fluconazol, flucytocine, griseofulvin, bifonazole, amorofine, mycostatin, itraconazole, terbenafine, terconazole, tolnaftate etc.

Antimicrobial agents like e.g. metronidazole, tetracyclines, oxytetracylines, peniciilins etc.

Antiemetics like e.g. metoclopramide, droperidol, haloperidol, promethazine etc.

Antihistamines like e.g. chlorpheniramine, terfenadine, triprolidine etc.

Antimigraine agents like e.g. dihydroergotamine, ergotamine, pizofylline etc.

Coronary, cerebral or peripheral vasodilators like e.g. nifedipine, diltiazem etc.

Antianginals such as, e.g., glyceryl nitrate, isosorbide dinitrate, molsidomine, verapamil etc.

Calcium channel blockers like e.g. verapamil, nifedipine, diltiazem, nicardipine etc.

Hormonal agents like e.g. estradiol, estron, estriol, polyestradiol, polyestriol, dienestrol, diethylstilbestrol, progesterone, dihydroprogesterone, cyprosterone, danazol, testosterone etc.

Contraceptive agents like e.g. ethinyl estradiol, lynestrenol, etynodiol, norethisterone, mestranol, norgestrel, levonorgestrel, desodestrel, medroxyprogesterone etc.

Antithrombotic agents like e.g. heparin, warfarin etc.

Diuretics like e.g. hydrochlorothiazide, flunarizine, minoxidil etc.

Antihypertensive agents like e.g. propanolol, metoprolol, clonidine, pindolol etc.

Corticosteroids like e.g. beclomethasone, betamethasone, betamethasone-17-valerate, betamethasone-dipropionate, clobetasol, clobetasol-17-butyrate, clobetasol-propionate, desonide, desoxymethasone, dexamethasone, diflucortolone, flumethasone, flumethasone-pivalte, fluocinolone acetonide, fluocinoide, hydrocortisone, hydrocortisone-17-butyrate, hydrocortisonebuteprate, methylprednisolone, triamcinolone acetonide, hacinonide, fluprednide acetate, alklometasone-dipropionate, fluocortolone, fluticason-propionte, mometasone-furate, desoxymethasone, diflurason-diacetate, halquinol, cliochinol, chlorchinaldol, fluocinolone-acetonide etc.

Dermatological agents like e.g. nitrofurantoin, dithranol, clioquinol, hydroxyquinoline, isotretionin, methoxsalen, methotrexate, tretionin, trioxalen, salicylic acid, penicillamine etc.

Steroids like e.g. estradiol, progesterone, norethindrone, levonorgestrel, ethynodiol, levonorgestrol, norgestimate, gestanin, desogestrel, 3-keton-desogesterel, demegestone, promethoestrol, testosterone, spironolactone and esters thereof etc.

Nitro compounds like e.g. amyl nitrates, nitroglycerine and isosorbide nitrate etc.

Opioids like e.g. morphine, buprenorphine, oxymorphone, hydromorphone, codeine, tramadol etc.

Prostaglandins such as, e.g., a member of the PGA, PGB, PGE or PGF series such as, e.g. minoprostol, dinoproston, carboprost, eneprostil etc.

Peptides like e.g. growth hormone releasing factors, growth factors (e.g. epidermal growth factor (EGF), nerve growth factor (NGF), TGF, PDGF, insulin growth factor (IGF), fibroblast growth factor (aFGF, bFGF etc.), somatostatin, calcitonin, insulin, vasopressin, interferons, IL-2 etc., urokinase, serratiopeptidase, superoxide dismutase, thyrotropin releasing hormone, lutenizing hormone releasing hormone (LH-RH), corticotrophin releasing hormone, growth hormone releasing hormone (GHRH), oxytocin, erythropoietin (EPO), colony stimulating factor (CSF) etc.

Other active substances of interest include ubiquinone (Coenzyme Q10), omega-3 fatty acids including fish oils containing such fatty acids, statins including simvastatin, lovastatin, atorvastatin, pravastatin, fluvastatin, rosuvastatin etc., fenofibrate.

Interesting examples are also prescription drugs like:

### Cardiovascular Drugs

Zocor®, Lipitor®, Prevachol®, Mevalotin®, Mevacor®, Lescol®, TriCor®, Norvasc®, Cozaar and Hyzaar®, Prinivil and Prinzide®, Diovan®/Co-Diovan®, Zestril®, Vasotech® and Vaseretic®, Lotensin®/Cibacen® and Lotrel®, Adalat®, Toprol-XL®/Seloken®, Tritace®/Delix®, Accupril® and Accuretic®, Avapro® and Avalide®, Plendil®, Monopril®, Blopress®, Atacand®, Tenormin®, Avapro®/Aprovel®, Coreg®, Altace®, Capoten®, Plavix®, Lovenox®/Clexane®, Fraxiparine®, ReoPro®, Panaldine®, Cordarone®

### Central Nervous System Drugs

Paxil/Seroxat®, Zolotoft®, Prozac®, Prozac Weekly® and Sarafem®, Effexor®, Wellbutrin®, Celexa®, Remeron®, Serzone®, Zyprexa®, Risperdal®, Seroquel®, Clozaril®/Leponex®, Neurontin®, Depaktoke®, Lamictal®, Topamax®, Tegretol®, Imitrex®/Imigran®, Zomig®, Maxalt®, Ambien®, Stilnox®, Ultane®/Sevorane®, Diprivan®, BuSpar®, Xanax®, Aricept®, Memantine®, Adderall®, Dystonia®, Botox®

### Anti-Infective Agents

Augmentin®, Cipro®/Ciprobay®, Zithromax®, Biaxin®, Levaquin® and Floxin®, Rocephin®, Primaxin®, Ceftin®/Zinnat®, Cravit®, Zosyn®/Tazocin®, Cefzil®, Tequin®, Tortaz®/Fortum®, Combivir®, Zerit®, Valtrex®, Epivir®, Zovirax®, Crixivan®, Viracept®, Viramune®, Kaletra®, Diflucan®, Lamisil®, Sporanox®

### Respiratory Drugs

ClaritinAllegra@, Telfast®, Zyrtec®, Flonase®/Flixonase®, Atrovent®, Nasonex®, Rhinocort®, Alesion®, Singulair®, Flovent®/Flixotide®, Advair®/Seretide®, Serevent®, Pulmicort®, Ventoline®, Combivent®, Synagis®, Mucosolvan®

### Gastrointestinal Drugs Prilosec®/Losec®, Prevacid®, Gaster®, Takepron®, Zantac®, Pantozol, Nexium, Protonix®, Aciphex®/Pariet®, Pepcid®, Axid®, Zoton®, Zofran®

### Cancer Drugs

### Taxol®, Taxotere®, Nolvadex®, Herceptin, Ellence®/Pharmorubicin®, Lupron®,

Zoladex®, Leuplin®, Casodex®, Intron A®, Peg-Intron® and Rebertron®, Rituxan®, Gemzar®, Paraplatin®, Camptosar®

### Antiarthritic Drugs/Analgesics

### Celebrex®, Vioxx®, Enbrel®, Remicade®, Voltaren®, Mobic®

### Duragesic®

### Ultram® and Ultrcet®

### Blood Disorder Treatments

### Procrit®/Eprex®, Epogen®, Epogin®, NeoRecormon®, Neupogen®, NovoSeven®

### Diabetes Drugs

### Glucophage@, Humulin Avandia®, Humalog®, Actos®, Amaryl®, Glucovance®, Glucophage XR®, Glucotrol XL®, Precose®/Glucobay®

### Bone Metabolism Regulators

### Fosamax®, Evista®, Miacalcin®, Actone®I, Aredia®

### Urinary Disorder Agents

### Harnal®, Proscar®, Cardura®, Flomax®, Detrol®

### Hormones

### Premarin®, Premphase® and Prempro®, Estraderm®, Synthroid®

### Immunosuppressive Agents

Neoral®/Sandimmun®, CellCept, Rapamune®, Tacrolimus e.g. Prograf®, Medrol®

### Multiple Sclerosis Drugs

### Avonex®, Betaseron®/Betaferon®, Rebif®, Copaxone®

### Biologicals

### Prevnar®, Engerix-B®, Infanrix®, Gamimune N®

### Sexual Dysfunction Drugs

### Viagra®

### Imaging Agents

### Iopamiron®, Omnipaque®, Magnevist®

### Ophthalmic Drugs

### Xalatan®, Trusopt® and Cosopt®

### Dermatological Drugs

### Accutane®/Roaccutan®, Cleocin®

### Growth Failure Therapies

### Genotropin®, Humatrope®

### Infertility Drugs

### Gonal-F®, Follistim (Puregon®

### Gaucher Disease Drugs

### Cerezyme®

### Obesity Drugs

### Xencial®

### Acromegaly Drugs

### Sandostatin®

### Contraceptives

### Depo-Provera®

Other interesting examples of active substances that are slightly soluble, sparingly soluble or insoluble in water are given in the following tables:

TABLE 1
Poorly-Soluble Drug Candidates

| Drug Name | Therapeutic Class | Solubility In Water |
|---|---|---|
| Alprazolam | CNS | Insoluble |
| Amiodarone | Cardiovascular | Very Slightly |
| Amlodipine | Cardiovascular | Slightly |
| Astemizole | Respiratory | Insoluble |
| Atenolol | Cardiovascular | Slightly |
| Azathioprine | Anticancer | Insoluble |
| Azelastine | Respiratory | Insoluble |
| Beclomethasone | Respiratory | Insoluble |
| Budesonide | Respiratory | Sparingly |
| Buprenorphine | CNS | Slightly |
| Butalbital | CNS | Insoluble |
| Carbamazepine | CNS | Insoluble |
| Carbidopa | CNS | Slightly |
| Cefotaxime | Anti-infective | Sparingly |
| Cephalexin | Anti-infective | Slightly |
| Cholestyramine | Cardiovascular | Insoluble |
| Ciprofloxacin | Anti-infective | Insoluble |
| Cisapride | Gastrointestinal | Insoluble |
| Cisplatin | Anticancer | Slightly |
| Clarithromycin | Anti-infective | Insoluble |
| Clonazepam | CNS | Slightly |
| Clozapine | CNS | Slightly |
| Cyclosporin | Immunosuppressant | Practically Insoluble |
| Diazepam | CNS | Slightly |
| Diclofenac sodium | NSAID | Sparingly |
| Digoxin | Cardiovascular | Insoluble |
| Dipyridamole | Cardiovascular | Slightly |
| Divalproex | CNS | Slightly |
| Dobutamine | Cardiovascular | Sparingly |
| Doxazosin | Cardiovascular | Slightly |
| Enalapril | Cardiovascular | Sparingly |
| Estradiol | Hormone | Insoluble |
| Etodolac | NSAID | Insoluble |
| Etoposide | Anticancer | Very Slightly |
| Famotidine | Gastrointestinal | Slightly |
| Felodipine | Cardiovascular | Insoluble |
| Fentanyl citrate | CNS | Sparingly |
| Fexofenadine | Respiratory | Slightly |
| Finasteride | Genito-urinary | Insoluble |
| Fluconazole | Antifungal | Slightly |
| Flunosolide | Respiratory | Insoluble |
| Flurbiprofen | NSAID | Slightly |
| Fluvoxamine | CNS | Sparingly |
| Furosemide | Cardiovascular | Insoluble |
| Glipizide | Metabolic | Insoluble |
| Glyburide | Metabolic | Sparingly |
| Ibuprofen | NSAID | Insoluble |
| Isosorbide dinitrate | Cardiovascular | Sparingly |
| Isotretinoin | Dermatological | Insoluble |
| Isradipine | Cardiovascular | Insoluble |
| Itraconzole | Antifungal | Insoluble |
| Ketoconazole | Antifungal | Insoluble |
| Ketoprofen | NSAID | Slightly |
| Lamotrigine | CNS | Slightly |
| Lansoprazole | Gastrointestinal | Insoluble |
| Loperamide | Gastrointestinal | Slightly |
| Loratadine | Respiratory | Insoluble |
| Lorazepam | CNS | Insoluble |
| Lovastatin | Cardiovascular | Insoluble |
| Medroxyprogesterone | Hormone | Insoluble |
| Mefenamic acid | Analgesic | Slightly |
| Methylprednisolone | Steroid | Insoluble |
| Midazolam | Anesthesia | Insoluble |
| Mometasone | Steroid | Insoluble |
| Nabumetone | NSAID | Insoluble |
| Naproxen | NSAID | Insoluble |
| Nicergoline | CNS | Insoluble |
| Nifedipine | Cardiovascular | Practically Insoluble |
| Norfloxacin | Anti-infective | Slightly |
| Omeprazole | Gastrointestinal | Slightly |
| Paclitaxel | Anticancer | Insoluble |
| Phenytoin | CNS | Insoluble |
| Piroxicam | NSAID | Sparingly |
| Quinapril | Cardiovascular | Insoluble |
| Ramipril | Cardiovascular | Insoluble |
| Risperidone | CNS | Insoluble |
| Saquinavir | Protease inhibitor | Practically insoluble |
| Sertraline | CNS | Slightly |
| Simvastatin | Cardiovascular | Insoluble |
| Terbinafine | Antifungal | Slightly |
| Terfenadine | Respiratory | Slightly |
| Triamcinolone | Steroid | Insoluble |
| Valproic acid | CNS | Slightly |
| Zolpidem | CNS | Sparingly |

TABLE 2
Poorly-Soluble Drugs with Low Bioavailability

| | | Solubility In | |
|---|---|---|---|
| Drug Name | Indication | Water | Bioavailability |
| Astemizole | Allergic Rhinitis | Insoluble | Low-moderate |
| Cyclandelate | Peripheral vascular | Insoluble | Low |
| | disease | | |
| Perphenazine | Psychotic disorder | Insoluble | Low |
| Testosterone | Androgen | Insoluble | Low |
| | Replacement | | |
| | Therapy | | |
| Famotidine | GERD | Slightly soluble | Low (39-50%) |
| Budesonide | Allergic Rhinitis | Sparingly soluble | Low (~15%) |
| Mesalamine | Irritable Bowel | Slightly soluble | Low (~20%) |
| | Syndrome | | |
| Clemastine | Allergic Rhinitis | Slightly soluble | Low (~39%) |
| fumarate | | | |
| Buprenorphine | Pain | Slightly soluble | Low (<30%) |
| Sertraline | Anxiety | Slightly soluble | Low (<44%) |
| Auranofin | Arthritis | Slightly soluble | Low (15-25%) |
| Felodipine | Hypertension | Insoluble | Low (15%) |
| Isradipine | Hypertension | Insoluble | Low (15-24%) |
| Danazol | Endometriosis | Insoluble | Low |
| Loratadine | Allergic Rhinitis | Insoluble | Low |
| Isosorbide | Angina | Sparingly soluble | Low (20-35%) |
| dinitrate | | | |
| Fluphenazine | Psychotic disorder | Insoluble | Low (2-3%) |
| Spironolactone | Hypertension, Edema | Insoluble | Low (25%) |
| Biperiden | Parkinson's disease | Sparingly soluble | Low (29-33%) |
| Cyclosporin | Transplantation | Slightly soluble | Low (30%) |
| Norfloxacin | Bacterial Infection | Slightly soluble | Low (30-40%) |
| Cisapride | GERD | Insoluble | Low (35-40%) |
| Nabumetone | Arthritis | Insoluble | Low (35%) |
| Dronabinol | Antiemetic | Insoluble | Low 10-20%) |
| Lovastatin | Hyperlipidemia | Insoluble | Low (~5%) |
| Simvastatin | Hyperlipidemia | Insoluble | Low (<5%) |

The amount of active substance incorporated in a tablet may be selected according to known principles of pharmaceutical formulation. In general, the dosage of the active substance present in a tablet according to the invention depends inter alia on the specific drug substance, the age and condition of the patient and of the disease to be treated.

In a specific embodiment of the invention the therapeutically, prophylactically and/or diagnostically active substance is solid at ambient temperature. However, this is not an absolute requirement, it may also be liquid at room temperature. The active substance may also be present in the form of a dispersion of the active substance in the pharmaceutically acceptable liquid formulation, or the active substance may be present in the form of an emulsion including a SMEDDs (self microemulsifying drug delivery system).

As mentioned above, the active substance may be dispersed in the pharmaceutically acceptable liquid formulation. In a specific embodiment, the active substance is at least partly dissolved in the pharmaceutically acceptable liquid formulation and/or it is at least partly present in an amorphous form.

### Other Aspects of the Invention

The invention also relates to a method for the preparation of a tablet comprising the steps of:

i) preparation of a loadable tablet as defined in any of claims 1-32 optionally comprising one or more therapeutically, prophylactically and/or diagnostically active substances,
ii) loading the loadable tablet obtained from step i) with a pharmaceutically acceptable liquid formulation as defined in any of claims 33-59 optionally comprising one or more therapeutically, prophylactically and/or diagnostically active substances for at time period that is sufficient to saturate the loadable tablet with the pharmaceutically acceptable liquid formulation.

The loading of the loadable tablet with the pharmaceutically acceptable liquid formulation optionally comprising one or more therapeutically, prophylactically and/or diagnostically active substances is typically performed by spraying or it is performed by placing the loadable tablet in an excess of the pharmaceutically acceptable liquid formulation optionally comprising one or more therapeutically, prophylactically and/or diagnostically active substances.

In the method mentioned above, the time period in step ii) is normally at the most about 60 min such as, e.g., at the most 45 min or at the most 30 min for an amount of loadable tablets corresponding to 1 kg (and corresponding time periods for batches having another weight than 1 kg).

The invention is further illustrated in the following non-limiting examples.

### EXAMPLES

### Example 1

### Preparation of Loadable Tablets and Properties Thereof

Six tablet compositions were manufactured based on the oil absorption materials Aeroperl 300 (Silicon dioxide, Degussa), Neusilin US2 (magnesium aluminium metasilicate, Fuji Chemical Industry) Avicel (microcrystalline cellulose, FMC) and Fujicalin SG, (dibasic calcium phosphate anhydrous, Fuji Chemical Industry).

### Composition 1

| | |
|---|---|
| Neusilin US2 | 99% |
| Magnesium stearate | 1% |

### Composition 2

| | |
|---|---|
| Avicel PH102 | 99% |
| Magnesium stearate | 1% |

### Composition 3

| | |
|---|---|
| Aeroperl 300 | 80% |
| PEG 6000 | 19% |
| Magnesium stearate | 1% |

### Composition 4

| | |
|---|---|
| Aeroperl 300 | 55% |
| Avicel PH 101 | 44% |
| Magnesium stearate | 1% |

### Composition 5

| | |
|---|---|
| Avicel PH 102 | 99% |
| Magnesium stearate | 1% |

### Composition 6

| | |
|---|---|
| Fujicalin | 99% |
| Magnesium stearate | 1% |

Magnesium stearate was blended with the remaining constituents in a Turbula blender for 3 minutes. The tablets were compressed on a single punch tabletting machine Diaf TM20. Tablet size: 9 mm round compound cup.

The tablets were placed in corn oil for 24 hours. The absorption of oil was completed within the first hour.

Tablets of composition 5 were loaded with with Imwitor 308, Sasol (glyceryl monocaprylate) with 10% dissolved Simvastatin. The loading with oil was performed at a temperature over melting point of Imwitor 308 (m.p. 35° C.) correspondig to 40° C.

### Composition 1.

TABLE 1
Oil absorption capacity of tablets containing Neusilin US2. (composition 1)

| | | Tablet | Oil | Oil |
|---|---|---|---|---|
| | Tablet core | core incl. | absorbed, | absorbed |
| Tablet no. | weight, mg | oil mg | mg | % |
| 1 | 142 | 367 | 225 | 61.3 |
| 2 | 139 | 364 | 225 | 61.8 |
| 3 | 143 | 369 | 226 | 61.2 |
| 4 | 144 | 367 | 223 | 60.8 |
| 5 | 142 | 370 | 228 | 61.6 |
| 6 | 150 | 370 | 220 | 59.5 |
| Mean | 143 | 368 | 224.5 | 61.0 |

The tablet hardness was determined by Schleuninger 8M tablet hardness tester.

TABLE 2
Tablet hardness before and after loading with oil (composition 1)

| Mean tablet hardness | Mean tablet hardness |
|---|---|
| before oil loading, N | after oil loading, N |
| 38 | 34 |

The disintegration time was exceeding 24 hours before and after loading with oil.

The disintegration time was decreased to less than 15 min. by addition of Ac-di-sol in a concentration of 1% (before loading) and reduced to 5 hours after oil loading. Ac-di-sol (croscarmellose sodium, FMC) is a superdisintegrant which does not affect the oil absorption capacity of Neusilin.

The porosity of the tablets before loading is calculated on basis of the density of the tablet ρₜ and the “true density” ρₛ of the ingredients. The porosity ε of the tablet is calculated according to the Equation 1.

$$ɛ = 1 - \frac{{ρ}_{t}}{{ρ}_{s}} . \qquad Equation 1$$

The density of the tablet is based on the ratio between weight and volume of the tablet. The “true density” of the ingredients is based on the gas pycnometrical density determined in helium using Micromeritics Accupyc 1330.

The maximum loading capacity of corn oil on weight basis is calculated according to Equation 2.

$$loading capcity w \text{/} w \% = \frac{ɛ}{ɛ + ( 1 + ɛ ) \frac{{ρ}_{s}}{{ρ}_{l}}} 100 \qquad Equation 2$$

The density of corn oil, ρₗ=0.92 g/cm³

TABLE 3
Utilization of oil loading capacity (composition 1).

| Porosity of the tablet | Max. oil loading | Measured oil loading |
|---|---|---|
| % | Capacity % | % |
| 80 | 63 | 61 |

### Composition 2

TABLE 4
Oil absorption capacity of tablets with Avicel (composition 2)

| | | Tablet | Oil | Oil |
|---|---|---|---|---|
| | Tablet core | core incl. | absorbed, | absorbed |
| Tablet no. | weight, mg | oil mg | mg | % |
| 1 | 232 | 349 | 117 | 33.52 |
| 2 | 229 | 351 | 1.22 | 34.76 |
| 3 | 230 | 351 | 121 | 34.47 |
| 4 | 229 | 349 | 120 | 34.38 |
| 5 | 229 | 353 | 124 | 35.13 |
| 6 | 230 | 349 | 119 | 34.10 |
| Mean | 230 | 350 | 121 | 34.39 |

The tablet hardness is determined by Schleuninger 8M tablet hardness tester.

TABLE 5
Tablet hardness before and after loading withoil (composition 2)

| Mean tablet hardness | Mean tablet hardness |
|---|---|
| before oil loading, N | after oil loading, N |
| 33 | 32 |

TABLE 6
Utilization of oil loading capacity (composition 2)

| Porosity of the tablet | Max. oil loading | Measured oil loading |
|---|---|---|
| % | Capacity % | % |
| 48 | 35 | 34 |

### Composition 3

TABLE 7
Oil absorption capacity of tablets with Aeroperl/PEG 6000
(composition 3)

| | | Tablet | Oil | Oil |
|---|---|---|---|---|
| | Tablet core | core incl. | absorbed, | absorbed |
| Tablet no. | weight, mg | oil mg | mg | % |
| 1 | 105 | 222 | 117 | 52.7 |
| 2 | 108 | 226 | 118 | 52.2 |
| 3 | 113 | 230 | 117 | 50.9 |
| 4 | 106 | 228 | 122 | 53.5 |
| 5 | 126 | 232 | 106 | 45.7 |
| 6 | 110 | 227 | 117 | 51.5 |
| Mean | 111.3 | 227.5 | 116.2 | 51.1 |

The tablet hardness is determined by Schleuninger 8M tablet hardness tester.

TABLE 8
Tablet hardness before and after loading with oil (composition 3)

| Mean tablet hardness | Mean tablet hardness |
|---|---|
| before oil loading, N | after oil loading, N |
| 15 | 10 |

TABLE 9
Utilization of oil loading capacity (composition 3)

| Porosity of the tablet | Max. oil loading | Measured oil loading |
|---|---|---|
| % | Capacity % | % |
| 70 | 54 | 51 |

### Composition 4

TABLE 10
Oil absorption capacity of tablets with Aeroperl/Avicel
(composition 4)

| | | Tablet | Oil | Oil |
|---|---|---|---|---|
| | Tablet core | core incl. | absorbed, | absorbed |
| Tablet no. | weight, mg | oil mg | mg | % |
| 1 | 192 | 324 | 132 | 40.7 |
| 2 | 198 | 329 | 131 | 39.8 |
| 3 | 204 | 329 | 125 | 38.0 |
| 4 | 193 | 325 | 132 | 40.6 |
| 5 | 193 | 325 | 132 | 40.6 |
| Mean | 196 | 326 | 130 | 39.9 |

TABLE 11
Tablet hardness before and after loading with oil (composition 4)

| Mean tablet hardness | Mean tablet hardness |
|---|---|
| before oil loading, N | after oil loading, N |
| 30 | 27 |

TABLE 12
Tablet disintegration time before and after loading with oil (composition 4)
Compared to composition 3 the tabletting properties and tablet hardness
were improved by addition of Avicel PH101 instead of PEG 6000.

| Mean disintegration time | Mean disintegration time |
|---|---|
| before oil loading, min | after oil loading, min |
| 2 | 1 |

### Composition 5

TABLE 13
Oil absorption capacity of tablets with Avicel. loaded with a 10% solution
of Simvastatin in Imwitor 308. (composition 5)

| | | Tablet | Oil | Oil |
|---|---|---|---|---|
| | Tablet core | core inch | absorbed, | absorbed |
| Tablet no. | weight, mg | oi lmg | mg | % |
| 1 | 229 | 338 | 109 | 32.2 |
| 2 | 229 | 337 | 108 | 32.0 |
| 3 | 229 | 337 | 108 | 32.0 |
| 4 | 229 | 339 | 110 | 32.4 |
| 5 | 230 | 338 | 108 | 31,9 |
| 6 | 229 | 337 | 108 | 32.0 |
| 7 | 229 | 338 | 109 | 32.2 |
| 8 | 229 | 338 | 109 | 32.2 |
| 9 | 229 | 339 | 110 | 32.4 |
| 10 | 228 | 339 | 111 | 32.7 |
| 11 | 230 | 340 | 110 | 32.4 |
| 12 | 230 | 338 | 108 | 31.9 |
| Mean | 229 | 338 | 109 | 32.2 |

TABLE 14
Tablet hardness before and after loading with a 10% solution
of Simvastatin in Imwitor 308 (composition 5)

| Mean tablet hardness | Mean tablet hardness |
|---|---|
| before oil loading, N | after oil loading, N |
| 35 | 32 |

TABLE 15
Tablet disintegration time before and after loading with oil (composition 5)

| Mean disintegration time | Mean disintegration time |
|---|---|
| before oil loading, min | after oil loading, min |
| 1 | 2 |

### Composition 6

TABLE 17
Oil absorption capacity of tablets with Fujicalin loaded with corn oil
(composition 6).

| | | Tablet | Oil | Oil |
|---|---|---|---|---|
| | Tablet core | core incl. | absorbed, | absorbed |
| Tablet no. | weight, mg | oil mg | mg | % |
| 1 | 258 | 383 | 125 | 48.4 |
| 2 | 259 | 384 | 125 | 48.3 |
| 3 | 259 | 383 | 124 | 47.9 |
| 4 | 260 | 383 | 123 | 47.3 |
| 5 | 257 | 382 | 125 | 48.6 |
| 6 | 261 | 384 | 123 | 47.1 |
| Mean | 259 | 383.2 | 124.2 | 47.9 |

TABLE 18
Tablet hardness before and after loading with corn oil (composition 6)

| Mean tablet hardness | Mean tablet hardness |
|---|---|
| before oil loading, N | after oil loading, N |
| 42 | 20 |

TABLE 19
Tablet disintegration time before and after
loading with corn oil (composition 6)

| Mean disintegration time | Mean disintegration time |
|---|---|
| before oil loading, min | after oil loading, min |
| 2 | 6.1 |

### Conclusion

Porous tablets can be used as carriers for oily formulations such as oils, emulsions, microemulsions and semisolids liquefied at elevated temperature including drug substances as in liquid form or dissolved or dispersed in a liquid carrier. The oils can be applied to the tablets by conventional coating techniques (drums, perforated vessels or fluid bed). The feed rate of the oil should be adjusted to balance the rate of absorption of oil into the tablet cores.

The oil absorption capacity is determined by the porosity of the tablet core. The oil is filling the tablets voids close to saturation.

Any material, which provides tablets with porosities in the range of 30-90%, is applicable. Other materials than mentioned above may be applied as tablet core material, such as calcium carbonate, magnesium oxide preferable spray dried materials with satisfactory flowability and high specific surface area. The disintegration time of the tablets might be adjusted by addition of conventional tablet disintegrants and used in formulation of immediate release tablets as well as controlled release matrix tablets.

### Examples of Porous Tablets Loaded with Active Substances (APIs)

### Example 2

### Specification of Core Tablets

| | | |
|---|---|---|
| Neusilin US2 | 93 | mg |
| Magnesium stearate | 1 | mg |
| Average tablet hardness: | 52 | N |
| Tablet diameter: | 8 | mm |

(compound cup)

The tablets were compressed on a single punch tabletting machine Diaf TM20.

### Specification of the Loaded Tablet (1 mg Tacrolimus)

Tacrolimus in a concentration of 0.95% is dissolved in polyethylene glycol 400 and sprayed on Neusilin US2 core tablet at ambient temperature in a coating vessel. The composition of the loaded 1 mg tablet is shown in Table 1 corresponding to a loaded tablet weight of 200 mg corresponding to a load of vehicle of 53% w/w. Average tablet hardness: 52 N

TABLE 20
Composition of a 1 mg tablet loaded
with a solution of tacrolimus in PEG 400

| Substance | mg |
|---|---|
| Tacrolimus | 1.00 |
| PEG 400 | 105.0 |
| Neusilin US 2 | 93 |
| Magnesium stearate | 1 |
| Total | 200 |

### Example 3

### Specification of Core Tablets

| | | |
|---|---|---|
| Neusilin US2 | 198 | mg |
| Magnesium stearate | 2 | mg |
| Average tablet hardness: | 42 | N |
| Tablet diameter: | 10 | mm |

(compound cup)

The tablets were compressed on a single punch tabletting machine Diaf TM20.

### Specification of the Loaded Tablet (20 mg Atorvastatin)

Atorvastatin in a concentration of 10% is dissolved in melted Imwitor 308 (glyceryl monocaprylate) at 40° C. and sprayed on Neusilin US2 core tablet heated to 35° C. in a coating vessel. The loaded tablets are cooled in a refrigerator after loading in order to solidify the vehicle.

The composition of the loaded 20 mg tablet is shown in Table 2 corresponding to a loaded tablet weight of 400 mg corresponding to a load of vehicle of 50% w/w. Average tablet hardness: 48 N

TABLE 21
Composition of a 20 mg tablet loaded with a
solution of atorvastatin in glyceryl monocaptylate.

| Substance | mg |
|---|---|
| Atorvastatin | 20.0 |
| Imwitor 308 | 180.0 |
| Neusilin US 2 | 198.0 |
| Magnesium stearate | 2.0 |
| Total | 400.0 |

### Example 4

### Specification of Core Tablets

| | | |
|---|---|---|
| Neusilin US2 | 351 | mg |
| Magnesium stearate | 2 | mg |
| Average tablet hardness: | 60 | N |
| Tablet shape: Oblong tablet | 9 × 19 | mm |

The tablets were compressed on a single punch tableting machine Diaf TM20.

### Specification of the Loaded Tablet (145 mg Fenofibrate)

Fenofibrate in a concentration of 35% is dissolved in a melted mixture of Polyethyleneglycol 6000 and Poloxamer 188 (70:30) at a temperature of 80° C. and and sprayed on Neusilin US2 core tablet heated in a coating vessel to a temperature of 70° C. The tablets are cooled in the coating vessel after loading to a temperature below the melting point (60° C.) of PEG and Poloxamer

The composition of the loaded 145 mg tablet is shown in Table 3 corresponding to a loaded tablet weight of 767 mg corresponding to a load of vehicle of 54% w/w. Average tablet hardness: 57 N

TABLE 22
Composition of a 145 mg tablet loaded with a solution
of fenofibrate in a melted mixture of PEG
6000 and Poloxamer 188 (70:30).

| Substance | mg |
|---|---|
| Fenofibrate | 145.0 |
| PEG 6000 | 188.4 |
| Poloxamer 188 | 80.8 |
| Neusilin US 2 | 350.8 |
| Magnesium stearate | 2.0 |
| Total | 767.0 |

### Example 5

### Specification of Core Tablets

| | | |
|---|---|---|
| Neusilin US2 | 84 | mg |
| Magnesium stearate | 1 | mg |
| Average tablet hardness: | 42 | N |
| Tablet diameter: | 7 | mm |

(compound cup)

The tablets were compressed on a single punch tableting machine Diaf TM20.

### Specification of the Loaded Tablet (10 mg Simvastatin)

Simvastatin in a concentration of 10% is dissolved in (MCT) Viscoleo on Neusilin US2 core in a coating vessel. The composition of the loaded 10 mg tablet is shown in Table 4 corresponding to a loaded tablet weight of 185 mg corresponding to a load of vehicle of 54% w/w.

TABLE 23
Composition of a 10 mg tablet loaded with a
solution of simvastatin in Viscoleo.

| Substance | mg |
|---|---|
| Simvastatin | 10.0 |
| Glyceryl monolaurate | 89.9 |
| Neusilin US 2 | 84.1 |
| Magnesium stearate | 1.0 |
| Total | 185.0 |

### Example 6

### Loading of Neusilin Tablets with Viscoleo

### Medium Chain Glyceride

### Tabletting Process

Neusilin tablets were compressed on a single punch tabletting machine Diaf TM20:

### Tablet Properties Before Loading

Tablet diameter: 9 mm
Tablet shape: Compound cup
Tablet weight: 134 mg
Tablet weight variation, Sᵣₑₗ: 1.6%
Tablet hardness: 51 N (determined on hardness tester Schleuniger M8)

### Loading Process (Loading Process)

50 g tablets were loaded with viscoleo in a lab-scale fluid bed Phast FB 100 using a coating module with top-spray

Atomization air flow: 1 m³ per hour
Fluidization air flow: 40 m³ per hour
Liquid feed rate: 2.5 g min
Coating time until saturation of the tablets: 30 min.
Weight increase: 67.5 g viscoleo.
Tablet Properties after Loading
Tablet weight: 305 mg (loading 56 w/w %)
Tablet hardness: 51 N
Tablet weight variation, Sᵣₑₗ: 5.1%

### CONCLUSION

Conventional coating equipment as a fluid bed is feasible for loading a liquid formulation on the porous tablets within a short processing time. The tablets quickly absorb the liquid applied by spraying on the tablet surface. The tablet hardness is not affected by the loading with the liquid. The weight variation is increased from 1.6% to 5.2% still being within acceptable limits related to dose variation when an active substance is incorporated.

1-63. (canceled)

64. A method for the preparation of a tablet comprising the step of loading an inert and loadable tablet with a pharmaceutically acceptable liquid formulation comprising one or more therapeutically, prophylactically and/or diagnostically active substances, wherein
the inert and loadable tablet (a) has a porosity of 30% v/v or more, (b) has a hardness of 20N or more, and (c) comprises one or more pharmaceutically acceptable porosity providing excipients selected from alkaline earth metal silicates and aluminum silicates at a concentration of about 50% w/w or more in the inert and loadable tablet.

65. The method of claim 64, wherein the loading step is performed by placing the inert and loadable tablet in an excess of the pharmaceutically acceptable liquid formulation comprising one or more therapeutically, prophylactically and/or diagnostically active substances.

66. The method of claim 64, wherein the loading step is performed for a time period sufficient to saturate the inert and loadable tablet with the pharmaceutically acceptable liquid formulation.

67. The method of claim 64, wherein the inert and loadable tablet is loaded with the pharmaceutically acceptable liquid formulation to a concentration of about 5% w/w or more (based on the total weight of the loaded tablet).

68. The method of claim 64, wherein the inert and loadable tablet is loaded with the pharmaceutically acceptable liquid formulation to a concentration of about 20% w/w (based on the total weight of the loaded tablet).

69. The method of claim 64, wherein the inert and loadable tablet is loaded with the pharmaceutically acceptable liquid formulation to a concentration of about 40% w/w or more (based on the total weight of the loaded tablet).

70. The method of claim 64, wherein (d) the inert and loadable tablet does not contain an antioxidant, (e) the inert and loadable tablet is free of diagnostically active substances, and (f) the inert and loadable tablet is not a foodstuff composition.

71. The method of claim 64, wherein the one or more pharmaceutically acceptable porosity providing excipients are present at a concentration of about 60% w/w or more in the inert and loadable tablet.

72. The method of claim 64, wherein the one or more pharmaceutically acceptable porosity providing excipients have a specific surface area (BET surface area) of at least 50 m²/g as measured by gas adsorption.

73. The method of claim 64, wherein the tablet has a hardness of 25 N or more.

74. The method of claim 64, wherein the tablet has a friability of about 5% or less.

75. The method of claim 64, wherein the metal is selected from the group consisting of magnesium, calcium, and mixtures thereof.

76. The method of claim 64, wherein the pharmaceutically acceptable porosity providing excipient is a metal silicate selected from the group consisting of magnesium silicate, calcium silicate, and mixtures thereof.

77. The method of claim 64, wherein the pharmaceutically acceptable porosity providing excipient is a metal silicate selected from the group consisting of bentonite, veegum, laponite, and mixtures thereof.

78. The method of claim 64, wherein the pharmaceutically acceptable porosity providing excipient comprises aluminum metasilicate.

79. The method of claim 64, wherein the pharmaceutically acceptable porosity providing excipient comprises magnesium aluminum metasilicate.

80. The method of claim 64, wherein the pharmaceutically acceptable liquid formulation has a viscosity of at the most about 600 mPa sec at a temperature of at the most about 150° C.

81. The method of claim 64, wherein the pharmaceutically acceptable liquid formulation comprises apricot oil, almond oil, avocado oil, castor oil, coconut fat, cocoa butter, corn oil, cotton seed oil, grape seed oil, jojoba oil, linseed oil, maize oil, olive oil, palm oil, peanut oil, persil oil, poppy seed oil, rape seed oil, sesame oil, soybean oil, sunflower oil, thistle seed oil, walnut oil, wheat germ oil, beef tallow, lard, tall oil, whale oil, or any combination of any of the foregoing.

82. The method of claim 64, wherein the inert and loadable tablet has a disintegration time of at the most 15 min as tested according to Ph. Eur.

83. A method for administering one or more therapeutically, prophylactically and/or diagnostically active substances to a patient, comprising orally administering to the patient a tablet prepared by the method of claim 64.
